(19) United States
(12) Patent Application Publication
(10) Pub. No.: US 2006/0099642 A1
(43) Pub. Date: May 11, 2006

(54) Solid-phase substrate for immobilizing biomolecules

(76) Inventors: Alexander Papra, Hamburg (DE); Thomas Kolzau, Hamburg (DE); Barbara Koeppen, Hamburg (DE)

Correspondence Address: DAVID TOREN, ESQ.;ABELMAN FRAYNE & SCHWAB, 666 THIRD AVENUE, NEW YORK, NY, 10017-5621, US

(21) Appl. No.: 10/517,501

(22) PCT Filed: Jun. 5, 2003
(86) PCT No.: PCT/EP03/05909

§ 371 Date: Sep. 13, 2005

(30) Foreign Application Priority Data
Jun. 10, 2002 (DE) 102 26 501.6

(51) Int. Cl. G01N 33/53; C07K 1/10; C12M 1/34

(52) U.S. Cl. 435/7.1; 530/412; 435/287.2; 536/121

(57) ABSTRACT

The invention relates to a solid phase substrate comprising at least one binding area, which is suited for immobilizing biomolecules. The solid phase substrate is characterized in that the substrate has, in the binding area, reactive binding sites at which polyol prefabricated by means of covalent bonds is immobilized.

The invention relates to a solid-phase substrate for immobilizing biomolecules of the introductory portion of claim 1, as well as to a method for immobilizing biomolecules in a sample of the introductory portion of claim 8.

The immobilization of biomolecules in solid-phase substrates is necessary, for example, for producing arrays, such as protein arrays, enzyme membranes, and reaction vessels coated with proteins.

Generic solid-phase substrates, suitable for enzyme membranes, consist, for example, of nitrocellulose, which is used especially for proteins because of its low price, its flexibility and its good bonding properties. The mechanisms, responsible for the bonding properties of biomolecules to nitrocellulose, are unknown. Presumably, however, they are based on the formation of hydrogen bonds as well as on hydrophobic and electrostatic interactions. In practice, the adsorption takes place simply by incubating the substrate in a solution containing biomolecules and subsequently drying.

In this connection, it is known (Nguyen V K et al. 1968, Stabilization of dry immobilized acetylcholine esterase on nitrocellulose membranes for rapid colorimetric screening of its inhibitors in water and biological fluids, Analytical Letters 31(14), 2457) that biomolecules may be immobilized in the presence of polyols such as tetrahalose, in order to prevent denaturing of the biomolecules during the drying with the objective of largely maintaining the natural conformation of the biomolecules immobilized on the substrate so that, for example, immobilized enzymes maintain their catalytic activity or antibodies their specific immunological properties. The polyols, used in a particular case, are also referred to as protective substances.

Moreover, the polyols can be absorbed on the nitrocellulose membrane simultaneously with as well as also after the biomolecules, which are to be immobilized. Since the functional groups of polyols, namely the OH groups, are similar to those of many biomolecules (such as the alcoholic amino acids tyrosine and serine), it may be assumed that polyols, like biomolecules, are adsorbed nonspecifically on the surface of the nitrocellulose membrane.

It is a disadvantage of these generic substrates that the polyols, absorbed on the substrate and not bonded covalently, may go into solution during the later use of the substrate and may affect the subsequent experiment. As a rule, therefore, it is necessary to wash the dried substrate carefully before any further use or before it is worked up.

It is an object of the invention to create a substrate and a method, with which the disadvantage, described above, can be countered.

This objective is accomplished with a solid-phase substrate with a bonding area, which is suitable for immobilizing biomolecules, the substrate having the characterizing distinguishing features of claim 1, as well as with a method, which has the characterizing distinguishing features of claim 8.

In accordance with claim 1, the inventive solid-phase substrate has reactive bonding sites in the bonding area, pre-synthesized polyols being immobilized at a portion thereof by means of covalent bonds. Suitable substrates may consist, for example, of plastic or also, of course, of glass. Usually, the substrates are two dimensional, such as slides or microscope slides. Other substrates, such as the walls of reaction vessels or the like, are also, of course, conceivable. Basically, all meaningful substrates, within the scope of the immobilization of biomolecules addressed here, are to be covered by the invention.

The separate adsorption of the polyol, required for generic substrates, becomes unnecessary when the inventive substrate is used. It is therefore not necessary to add any polyols during the whole of the further processing process. Moreover, the substrate does not have to be washed before a further use, since the polyol present is bonded covalently and not only adsorbed. Therefore, an effect on a subsequent experiment by polyols going into solution need not to be feared. Such a pre-manufactured solid-phase substrate, coated with covalently bonded polyols, is suitable particularly for spotting with protein or nucleic acid probes or samples and is therefore particularly suitable for producing bioarrays.

In a preferred embodiment, provisions are made so that the bonding area is designed for immobilizing proteins. The reactive bonding sites in the bonding area must therefore have functional groups suitable for covalently bonding proteins, such as ester-active groups, so that they can enter into covalent bonds with amino groups. Of course, other bonding sites are also conceivable. Within the scope of the invention, bonding sites are understood to be essentially functional groups, which are coupled to the substrate in the bonding area and are able to enter into a bond with the respective biomolecules and the polyols.

In further inventive embodiments, the bonding area may be designed so that it is suitable for bonding other biomolecules, such as sugars, lipids or nucleic acids. The conventional bonding sites, known to those skilled in the art, are suitable for this and will not be dealt with in greater detail here.

In a particularly preferred development, the substrate has polymer chains, which are coupled to its surface. Moreover, provisions are made so that the polymer chains, which are disposed at the substrate, carry the necessary, reactive bonding sites for coupling the biomolecules. In this case, the polyols, necessary for stabilizing the immobilized biomolecules, are also bonded to the polymer chains.

One end of the polymer chains, which are not cross-linked, may be coupled to the surface of the substrate and the other end may protrude therefrom. However, cross-linking of the polymers with one another to different degrees up to a hydrogel is also conceivable. The use of such polymer chains increases the active surface area of the substrate and thus permits a larger quantity of polyols and/or biomolecules to be bonded.

In a preferred development, provisions are furthermore made to bond polyethylene glycol in the polymer chains. Such PEG-containing polymers shield the substrate surface, for example, against non-specific, non-covalent absorption of other proteins.

For producing the previously made-up substrate, polyols, such as monsaccharides, disacchararides or trisaccharides may be used, within the scope of the invention, as protective substances. For example, maltose, sucrose, raffinose or glucose are suitable. Basically, all substances are suitable, which can be coupled over OH groups to an activated surface of a substrate and are in a position to stabilize the three-dimensional conformation of biomolecules, such as proteins. The use of tetrahalose is particularly preferred.

In a preferred embodiment, provisions are made so that the solid phase substrate is a biochip, an enzyme chip, a protein array, a filter membrane, a microbead, a reaction vessel, a micro-channel system, a flow-through tube system, the tip of a pipette or a flow-through cannula.

The invention, however, is also to include a method for immobilizing biomolecules.

In accordance with claim 8, the inventive method sees to it that a sample is brought into contact with a solid phase substrate, which has at least one bonding area, which is suitable for immobilizing biomolecules and for which the immobilization takes place in the presence of a substrate, which is in a position to stabilize the three-dimensional conformation of the biomolecules, the substrate having reactive bonding sites in the bonding area and polyols being used as substance and being bonded by means of covalent bonds to a portion of the bonding sites in the bonding area of the solid phase substrate.

In a preferred development of the method, the biomolecules, which are to be immobilized, are proteins. The reactive bonding sites in the bonding area must therefore have functional groups, which are suitable for bonding proteins covalently.

In further inventive developments, the biomolecules, which are to be immobilized, may also be sugars, lipids or nucleic acids. The reactive bonding sites in the bonding area may have the functional groups, which are suitable for these cases and are known to those skilled in the art.

Known methods of immobilizing biomolecules, for which proteins, for example, are used as biomolecules, comprise essentially the following steps:
1. A protein-containing solution is brought into contact with an activated substrate. The substrate may, for example, be a filter, a membrane, a chip or a reaction vessel, to name but a few of the usual devices.
   The usual substrates have reactive bonding sites, which can enter into covalent bonds with functional groups of the proteins, which are to be bonded. Upon contact with the substrate, the proteins are bonded covalently to the reactive bonding sites and immobilized at the substrate in this way.
2. Optionally, the substrate is then washed.
3. In a next step, the bonding sites on the substrate are blocked, for example, by the addition of a strongly nucleophilic agent, such as a short-chain amine.
4. The substrate is then washed once again, this last washing buffer containing a high concentration of a protective substance, which is in a position two stabilize the three-dimensional conformation of proteins. In the known method, this substance is trehalose, a polyol.
5. The substrate is then dried in the usual way, the trehalose being absorbed from the washing buffer onto the substrate.

In contrast to this method from the state of the art, the blocking step with a strongly nucleophilic agent and the last washing step, during which a polyol is adsorbed on the substrate, can be omitted. In distinction from the state of the art, the polyol is moreover not absorbed on the substrate surface in the case of the inventive method. Instead, it is covalently bonded to this surface, just as are the biomolecules.

The covalent immobilization of the polyols on the substrate does not have a disadvantageous effect on the function of the polyols as a protective substance for the biomolecules. However, contrary to the already known method, it is a significant advantage and difference that the polyols, because they are coupled permanently to the substrate, no longer interfere with a later use. An expensive washing step no longer has to be provided before the substrate, with the biomolecules immobilized thereon, is worked up.

In a preferred development of the inventive method, provisions are made so that the polyols are bonded covalently to the solid phase substrate over polymer chains, which are disposed in the bonding area and carry the reactive bonding sites. The use of such polymer chains increases the active surface area of the substrate and thus permits more polyols and/or biomolecules to be bonded.

It is especially preferred if the polyols and the biomolecules are bonded simultaneously to the solid phase substrate. This can be achieved most easily in that, for example, a protein solution, used for the immobilization, is mixed with the polyol. The bonding sites are then coupled proportionately with the polyols and the biomolecules or proteins, depending on the polyol concentration used.

In a solution, which contains, for example, proteins as biomolecules, suitable protein concentrations range from 1 to 100 g/l. Particularly suitable polyol concentrations range from 5 to 50 g/l.

In a further, particularly preferred development of the method, provisions are made so that the solid phase substrate used already contains pre-synthesized, bonded polyols.

Such a solid phase, provided with pre-synthesized, covalently bonded polyols, is suitable particularly for spotting with protein or nucleic acid probes or samples. Possible areas of application are, for example, the large-scale, serial production of standardized bioarrays, for which the immobilized biomolecules must be protected, so that they are not denatured while the bioarray is stored before use.

In a preferred development of the method, provisions are made so that the solid phase substrate is dried after it is brought into contact with the sample. In this development, the protective effect of the polyols, used as protective substances, comes to the fore particularly clearly, since drying of a substrate, which is coated with biomolecules and does not have any polyols, usually leads to the denaturation of the biomolecules.

In a further preferred embodiment of the method, provisions are made so that the polymer chains have additional PEG in the bonding area. Such PEG-containing polymers shield the substrate surface, for example, against non-specific absorption of other biomolecules.

Monosaccharide, disaccharides or trisaccharides, for example, are polyols, which can be used within the scope of the invention. Maltose, sucrose, raffinose, galactose or glucose, for example, is suitable. The preceding listing is given only by way of example and is not, by any means, complete. Basically, all substances are suitable, which can be coupled over suitable functional groups, such as OH groups, to an activated surface of a substrate and are in a position to stabilize the three-dimensional conformation of biomolecules, especially of proteins. The use of tetrahalose, a polyol, is particularly preferred. As shown by experiments of the applicant, it has a very stabilizing effect on the biomolecules immobilized on the substrate.

In a preferred embodiment of the method, provisions are made so that the solid phase substrate is a biochip, an enzyme chip, a protein array, a filter membrane, a microbead, a reaction vessel, a micro-channel system, a flow-through tube system, a tip of a pipette or a flow-through cannula.

Some of the substrates, used pursuant to the invention, can be produced generally by an in situ technique by photo-initiated graft polymerization, described, for example, by Ulbricht et al. in Colloids and Surfaces, vol. 138, 1998, page 353.

For preparing the inventive substrates, monomers, to which the polyols are bonded, are copolymerized for the method mentioned with monomers, which have the bonding sites for the proteins, such as ester-active groups. The desired ratio of polyol-bonding sites to protein-bonding sites in the polymers can be adjusted particularly easily by adjusting the starting concentrations of the different monomers. The copolymerization of other monomers with different groups into the polymers is, of course, also conceivable. For example, monomers, which contained PEG, could be copolymerized.

In the following, the invention is explained in even greater detail by means of an example. The example relates to a protocol, which can be carried out within the scope of the invention and for which a protein and trehalose are brought simultaneously into contact with the substrate.

### EXAMPLE

### Immobilization of Trypsin on a Carboxylated Nylon Membrane

The following procedure is used to obtain membranes, which are coated with active trypsin, as used, for example, in the “Double Parallel Digestion” Method (Bienvenut et al., Anal. Chem., vol. 71, 1999, pages 4800-4807) for protein identification and which, moreover, can be dried.

Abbreviations Used:

PP—phosphate buffer (0.1 M, pH 4.8)
PVDF=polyvinylidene fluoride
EDC=1-ethyl-3-(3-dimethylaminopropyl) carbodiimide
NHS=N-hydroxysuccinimide
PBS=phosphate-buffered physiological salt solution (pH 74)
Triton-X=nonionic surfactant
Tween=non-ionic surfactant

1. A carboxylated PVDF membrane (Millipore, Inc.) with an average pore size of 0.45 μm is incubated for 10 minutes with PP and then activated for 12 minutes with EDC dissolved in PP (25 mg/ml) and with NHS dissolved in PP (10 mg/ml) in the ratio of 1:1.

2. Subsequently, the activated membrane is washed with PP and incubated for 120 minutes with a PP solution, which contains trypsin (2 μg/100 μl) as well as trehalose (50 g/l). Subsequently, the membrane is washed with PP.

3. The membrane is then incubated for 10 minutes with 20 μg/100 μl of methoxyethylamine in order to bond the trypsin and trehalose covalently to the membrane.

4. Subsequently, the membrane is washed three times with PP, which contains 0.05% Triton X and incubated for 10 minutes with PBS/Tween.

5. The last solution is then removed and the membrane is dried.

The membrane, charged with active trypsin by this method, is stable when stored in the dry state and can be transported or shipped easily.

1. Solid-phase substrate with at least one bonding area, which is suitable for immobilizing biomolecules, characterized in that the substrate has reactive bonding sites in the bonding area, pre-synthesized polyol being immobilized at a portion of these bonding sites by means of covalent bonds.

2. The solid-phase substrate of claim 1, characterized in that the bonding area is designed for immobilizing proteins.

3. The solid-phase substrate of claims 1, characterized in that the polymer chains, which carry reactive bonding sites, are disposed in the bonding area.

4. The solid-phase substrate of claim 3, characterized in that additional PEG is bonded in the polymer chains.

5. The solid-phase substrate of claim 1, characterized in that the polyol used is a monosaccharide, disaccharide or trisaccharide.

6. The solid phase substrate of claim 1, characterized in that the polyol used is trehalose.

7. The solid-phase substrate of claim 1, characterized in that the solid-phase substrate is a biochip, an enzyme chip, a protein array, a filter membrane, a microbead, a reaction vessel, a micro-channel system, a flow-through tube system, the tip of a pipette or a flow-through cannula.

8. Method for immobilizing biomolecules in a sample, for which the sample is brought into contact with a solid-phase substrate, which has at least one bonding area, which is suitable for immobilizing biomolecules, and for which the immobilization takes place in the presence of a substance, which is in a position to stabilize the three-dimensional confirmation of the biomolecules, characterized in that the substrate has reactive bonding sites in the bonding area, polyols are used as substance and the polyols are bonded covalently to a portion of the bonding sites during the method.

9. The method of claim 8, characterized in that the biomolecules are proteins.

10. The method of claim 8, characterized in that the polyols are bonded covalently to the solid-phase substrate over polymer chains, which are disposed in the bonding area and carry reactive bonding sites.

11. The method of claim 8, characterized in that polyols and biomolecules are bonded simultaneously to the solid-phase substrate.

12. The method of claim 8, characterized in that a solid-phase substrate is used, which already contains pre-synthesized, bonded polyols.

13. The method of claim 8, characterized in that the solid-phase substrate, after it has been brought into contact with the sample, is dried.

14. The method of claim 8, characterized in that the polymer chains have additional PEG in the bonding area.

15. The method of claim 8, characterized in that the polyol used is a monosaccharide, disaccharide or trisaccharide.

16. The method of claim 8, characterized in that the polyol used is trehalose.

17. The method of claim 8, characterized in that the solid phase substrate is a biochip, an enzyme chip, a protein array, a filter membrane, a microbead, a reaction vessel, a micro-channel system, a flow-through tube system, the tip of a pipette or a flow-through cannula.

18. (canceled)

19. Method of spotting biomolecules, comprising the step of using, as a slide, a solid-phase substrate with at least one bonding area, which is suitable for immobilizing biomolecules, and having reactive bonding sites in the bonding area, with pre-synthesized polyol being immobilized at a portion of these bonding sites by means of covalent bonds.
